(19) United States
(12) Patent Application Publication
(10) Pub. No.: US 2026/0315191 A1
(43) Pub. Date: Oct. 15, 2026

(54) ORTHODONTIC DEVICE

(71) Applicant: Yves Bruno, Toulon (FR)

(72) Inventor: Yves Bruno, Toulon (FR)

(21) Appl. No.: 19/483,736

(22) PCT Filed: Jun. 6, 2024
(86) PCT No.: PCT/EP2024/065612

§ 371 Date: Nov. 12, 2025

(30) Foreign Application Priority Data
Jun. 19, 2023 (FR) FR2306299

(51) Int. Cl. A61C 7/36; A61C 7/08; A61F 5/56

(52) U.S. Cl. CPC A61C 7/36; A61C 7/08; A61F 5/566

(57) ABSTRACT

The orthodontic device for the treatment of dental malocclusion, dental alignment, and/or OSAS, includes an upper tray and a mandibular tray, each configured to conform to the dental arch, which cooperate via at least one connecting element formed by the association of a peg with a fin having an opening. The upper tray cooperates with the mandibular tray by insertion of the peg into the fin opening, allowing longitudinal translational movement of the peg within the opening along a direction parallel to the outer face of the user's dental arch.

FIELD OF THE INVENTION

The present invention falls within the field of medical devices, particularly orthodontic devices intended to treat dental pathologies. More specifically, the invention relates to an orthodontic device of a dental orthosis type.

### BACKGROUND ART

Such a device is specifically intended for application in the treatment of dental malocclusion, in particular Class II or Class III dental malocclusion, as well as in the correction of dental alignment, and also in the treatment of obstructive sleep apnea syndrome (OSAS).

There are currently numerous dental orthosis devices making use of bilateral rods, often metallic or using springs, intended to treat malocclusion or OSAS.

However, these devices present, in part or as a whole, the following drawbacks:
The structural design of these devices does not permit the simultaneous treatment of both dental malocclusion and dental alignment problems with a single appliance and in parallel.
Combining them with another appliance for alignment correction is often impossible due to excessive intraoral bulk or structural and functional incompatibilities.
The presence of rods or springs, often metallic, on both sides of the dental arch, along the vestibular surfaces of the teeth, creates intraoral bulk and a risk of mucosal injury, particularly to the cheeks or near the patient's lips.
The overall volume occupied by the appliance in the patient's mouth causes significant discomfort during mandibular opening/closing and may limit or prevent full mouth opening.
Spring systems involving compression/decompression present a risk of breakage due to their rigidity and are limited by available compression length; the (de)compression travel may be ill-suited to the user's morphology or mouth-opening capacity.
Telescopic sliding tube-rod systems risk blockage of the telescopic motion and are limited by the fixed length of the tubes.
The rigidity of the materials used to connect and secure the upper maxilla to the lower mandible does not allow precise mandibular correction or sufficient lateral jaw flexibility.
In certain cases, correction of malocclusion, particularly in the anteroposterior direction, becomes impossible as soon as the mouth opens with a pronounced vertical movement.

### SUMMARY OF THE INVENTION

The purpose of the present invention is to overcome these technical drawbacks by proposing an orthodontic device (1) for the treatment of dental malocclusion and/or correction of dental alignment and/or treatment of obstructive sleep apnea syndrome (OSAS), characterized in that it comprises:
an upper dental tray (2) and a mandibular dental tray (3), each configured to conform at least partially to a user's dental arch;
the upper tray (2) being configured to cover at least one premolar (PS) and/or molar (MS) on each side of the user's upper arch (4);
the mandibular tray (3) configured to cover at least one premolar (PI) and/or molar (MI) and/or canine (CI) on each side of the lower arch, or mandible (5);
the upper tray (2) cooperating with the mandibular tray (3) via at least one connecting element (6) positioned along the vestibular faces of the user's teeth or along the palatal and lingual faces;
the connecting element (6) comprising the association of a peg (7), integral with either the upper tray (2) or the mandibular tray (3), and a fin (8) having an opening (81), integral respectively with the mandibular tray (3) or the upper tray (2);
the peg (7), projecting in a plane perpendicular to the vestibular faces or to the palatal and lingual faces of the user's teeth, being configured to pass through the opening (81) of the fin (8) and to be laterally retained therein;
the fin (8), being substantially triangular, having an opening (81); preferably said opening (81) being substantially oblong;
the fin (8) being oriented vertically between the upper tray (2) and the mandibular tray (3) in a plane parallel to the vestibular faces or to the palatal and lingual faces of the user's teeth;
the opening (81) extending between the upper tray (2) and the mandibular tray (3) along a longitudinal axis (L);
the upper tray (2) cooperating with mandibular tray (3) by insertion of the peg (7) into the opening (81) of the fin (8), thereby allowing longitudinal translational movement of the peg (7) within the opening (81) along a direction substantially parallel to longitudinal axis (L) extending between the upper trays (2) and the mandibular (3).

Thus, the device (1) enables the treatment of Class II and Class III dental malocclusions via its specific connecting element (6), and, if needed, dental alignment by appropriately configuring the upper tray (2) and the mandibular tray (3). This holds whether the mouth is open or closed, while remaining structurally simple, non-bulky in the labial zone, and minimizing the risk of mucosal injury.

In addition, in accordance with other aspects of the invention:
Said device (1) comprises two connecting elements (6) positioned on either side of the dental arch, in a plane parallel to longitudinal axis (L), extending between the upper tray (2) and the mandibular tray (3); preferably, both connecting elements (6) are positioned along the vestibular faces of the user's teeth.
Each connecting element (6) extends toward either the vestibular faces or the palatal and lingual faces of the user's teeth with a thickness (E) comprised between 0.8 mm and 1.2 mm, preferably 1 mm.

Accordingly, even comprising the two connecting elements (6), the device (1) remains of low volume in the user's mouth, either at the cheek mucosa when positioned on the vestibular side, or at the palate and lips when positioned on the palatal/lingual side. The device of the invention is minimally obtrusive intraorally and does not hinder or prevent jaw movement.

In one embodiment of the device, the fin (8), which is substantially triangular, comprises, on the one hand, a side (82) whose full length serves as an anchoring base (82) on either the upper tray (2) or the mandibular tray (3); and, on the other hand, another side (83) whose end is secured, by insertion of the peg (7) into the opening (81), to the mandibular tray (3) or, respectively, to the upper tray (2).

Consequently, the connecting element (6) joins the upper tray (2) and the mandibular tray (3) via the fin's anchoring base (82) and the peg's insertion into the fin opening (81). The connecting element (6) keeps the upper tray (2) and the mandibular tray (3) mutually secured.

In a preferred embodiment suitable for treating Class II or Class III malocclusion:
the fin (8) is substantially in the form of a right triangle comprising a hypotenuse (84) connected by two legs (82, 83) meeting at a substantially right angle (d), one of the legs serving as an anchoring base (82); and in that, on the one hand, the hypotenuse (84) extends between upper tray (2) and mandibular tray (3); and, on the other hand, the right angle (d) defines a first anchoring point (P1) on one of the trays (2 or 3), while the peg (7), inserted into the opening (81), defines a fixing point (P2) with the fin (8) on the other tray (3 or 2, respectively),
the anchoring point (P1) or fixing point (P2) is positioned on one of the trays (2 or 3) at the disto-vestibular cusp or mesio-vestibular cusp of the first molar, or on the vestibular face of the second premolar,.
the leg (83) of the fin (8) that does not serve as anchoring base (82) has an angular offset comprised between 0° and 20°, preferably 16°, relative to the right-angle anchoring point (P1) on the upper tray (2) or the mandibular tray (3).

This preferred embodiment enables mandibular propulsion or retropropulsion to treat Class II or Class III malocclusion and to act mechanically on OSAS, particularly by virtue of the device's connecting element. If necessary, dental alignment can also be corrected by the specific molding of the trays covering all or part of the user's teeth.

A device that is simple to design and manufacture and custom-adaptable to any jaw type, dental arch, or dentition can thusly treat three conditions either simultaneously or independently.

In another preferred feature, the peg (7) forms a monolithic piece with either the upper tray (2) or the mandibular tray (3), and/or said fin (8) with the opening (81) forms a monolithic piece with either the upper tray (2) or the mandibular tray (3).

The use of monolithic pieces simplifies manufacture and facilitates intraoral placement and removal. No complex attachment systems such as interlocking tubes, hooks, or springs, are required. The device can be easily inserted and removed by the patient, and cleaning is straightforward.

In a preferred embodiment, the peg (7) forms a monolithic piece with the upper tray (2) or the mandibular tray (3), and/or said fin (8) with its opening (81) forms a monolithic piece with the upper tray (2) or the mandibular tray (3), thereby limiting overall bulk and volume in the mouth, and, improving comfort.

Advantageously, the device can be manufactured by thermoforming or by stereolithography.

These two processes are quick to implement, require few steps, can be carried out at a low cost, and yield a device in under one hour after taking the patient's dental impressions. On an industrial scale, the device is easy to produce using techniques well-known to a person skilled in the art, such as resin thermoforming and laser or mechanical cutting.

The device of the invention is therefore structurally simple to manufacture and position intraorally, non-intrusive for users, and provides effective treatment of Class II or Class II dental malocclusion in parallel with dental alignment issues and OSAS.

### BRIEF DESCRIPTION OF THE DRAWINGS

Further features and advantages will become apparent from the following detailed, non-limiting description with reference to the appended figures, in which:

FIG. 1 schematically illustrates the alignment of the teeth in the two dental arches in a Class I dental malocclusion, known as “dental normocclusion”;

FIG. 2A schematically illustrates the misalignment between the teeth of the two dental arches in a Class II dental malocclusion;

FIG. 2B schematically illustrates the misalignment between the teeth of the two dental arches in a Class III dental malocclusion;

FIG. 3 shows the orthodontic device (1) of the invention according to a first embodiment in which all of the user's teeth are covered by the upper tray (2) and the mandibular tray (3);

FIG. 4 shows the orthodontic device (1) of the invention according to another embodiment in which only part of the teeth of the user's lower maxilla are covered by the mandibular tray (3);

FIG. 5 shows the orthodontic device (1) of the invention shown in FIG. 4 positioned on a model of the user's dental arch;

FIG. 6 shows the upper tray (2) intended to be positioned on the maxilla of the user's upper arch before assembly with the peg (7), manufactured following a process of resin thermoforming, according to a manufacturing method of the orthodontic device (1) of the invention;

FIG. 7 shows the upper tray (2) of FIG. 6 after fixation and assembly with the peg (7);

FIG. 8 shows the mandibular tray (3) intended to be positioned on the user's lower arch according to the first embodiment of the orthodontic device (1) of FIG. 3;

FIG. 9 shows the mandibular tray in front view, positioned on a dental model of the user's lower maxilla, according to a variant of the embodiment of FIG. 4;

FIG. 10 shows a top view of the mandibular tray (3) of FIG. 8 before vertical folding of the fins (8), in which the fins present a 16° angular offset relative to the right-angle anchoring point (P1);

FIG. 11 schematically shows possible maxillary anchoring points for positioning the connecting element (6) of the orthodontic device (1) of the invention to treat Class II or III dental malocclusion and/or OSAS;

FIG. 12 schematically shows a profile view of the connecting element (6) of the orthodontic device (1) of the invention with maxillary anchoring on the disto-vestibular cusp of the first molar.

### DETAILED DESCRIPTION OF PARTICULAR EMBODIMENTS

The present invention relates to an orthodontic device (1) formed as a removable orthosis molded fully or partially to the shape of the teeth so as to engage with the user's dental arches and jaw.

The device (1) is used for the treatment of dental malocclusion, the correction of dental alignment, and/or the treatment of obstructive sleep apnea syndrome (OSAS).

The device (1) advantageously treats dental malocclusion, which consists of abnormal contact between the maxillary and mandibular teeth.

As shown in FIG. 1, Class I malocclusion, or “normocclusion,” i.e., normal occlusion, occurs when the mesio-buccal cusp of the upper molar (MS) occludes with the groove of the lower molar (MI).

According to the invention, the device (1) treats Class II and Class III dental malocclusions in which the upper and lower molars are incorrectly aligned with each other.

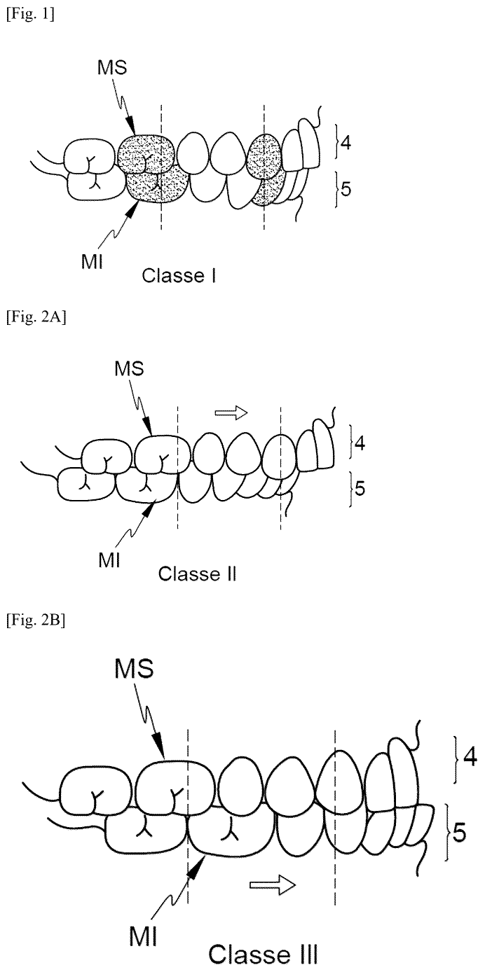

As shown in FIG. 2A, Class II dental malocclusion occurs when the teeth of the lower, or mandibular, arch (5) are positioned too far posteriorly relative to the teeth of the upper, or maxillary, arch (4). In a Class II dental malocclusion, the upper molar (MS) is located more anteriorly than the lower molar (MI), as are the remaining teeth of the maxilla, as illustrated by the arrow.

As shown in FIG. 2B, a Class III dental malocclusion occurs when the lower teeth are positioned too far anteriorly relative to the upper teeth, as illustrated by the arrow. In a Class III dental malocclusion, the lower molar (MI) is located more anteriorly than the upper molar (MS), as are the remaining teeth of the mandible, as illustrated by the arrow.

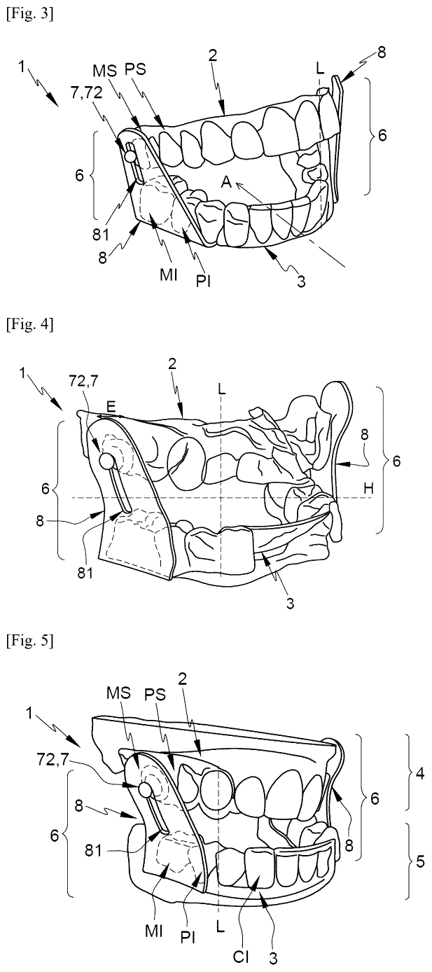

When placed in a patient's mouth, the device (1) can:
Treat a Class II dental malocclusion by generating mandibular propulsion, i.e., anterior advancement of the mandible toward the patient's lips along an anteroposterior axis A extending from the lips toward the patient's trachea, as shown in FIG. 3; or
Treat a Class III dental malocclusion by generating mandibular retropropulsion, i.e., posterior movement of the mandible toward the trachea along anteroposterior axis A extending from the lips toward the patient's trachea, in order to return to a “dental normocclusion”.

Thus, when placed in the patient's mouth, the device (1) treats Class II or Class III dental malocclusions by mechanical constraint and correction, through mandibular propulsion or retropropulsion, respectively, relative to the maxilla.

Advantageously, the device (1) makes it possible, simultaneously with the treatment of Class II or III dental malocclusion, to act mechanically on mandibular advancement and thus to mechanically treat OSAS, while also enabling, if necessary, correction of dental alignment through the configuration of the dental trays (2 or 3).

Indeed, as shown in FIGS. 3 to 5, the device (1) comprises an upper dental tray (2) and a mandibular dental tray (3).

According to the invention, the upper tray (2) and the mandibular tray (3) are each configured to conform at least partially to the user's dental arch. The upper tray (2) and the mandibular tray (3) are curved and generally U-shaped to fit the user's dental arches, in particular to fit the user's teeth and dentition.

In a first embodiment shown in FIG. 3, the upper tray (2) and the mandibular tray (3) conform to and cover all of the teeth of the user's dental arch. This configuration allows rapid anchorage and improved mechanical grip of the device (1) on the teeth, facilitates placement in the patient's mouth, and reduces the risk of mechanical breakage of the device, for example, during mastication or mouth opening.

In another embodiment, the upper tray (2) and the mandibular tray (3) are configured to conform to and cover only part of the teeth of the patient's dental arch.

For example, as shown in FIGS. 4 and 5, the user's incisors and part of the canines are not fully covered by the mandibular tray (3). In this case, the incisors and canines contact the mandibular tray (3) only along their lingual faces facing the patient's tongue, and can be mechanically advanced forward, toward the lips and vestibular surfaces of the teeth. This configuration enables correction of dental alignment by advancing the user's lower incisors and canines. Thus, the configuration and shape of the upper tray (2) and the mandibular tray (3) of the device (1), which fully or partially cover the patient's teeth, also enables, if necessary, dental alignment correction.

According to the invention, the upper tray (2) is specifically configured to cover at least one of the user's premolars (PS) and/or molars (MS), right (D) and left (G), of the upper arch (4), also referred to as the maxilla (4).

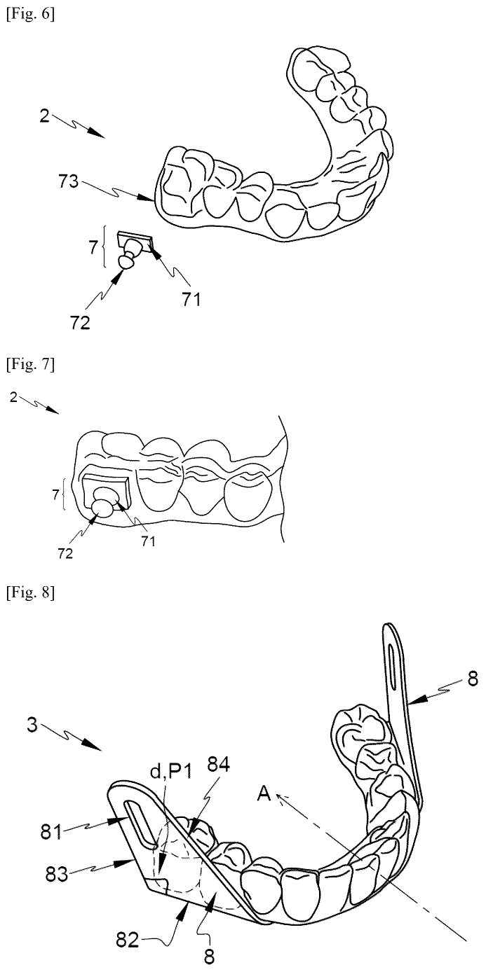

In the context of this invention, right (D) and left (G) refer to the sides of the dental arch located on either side of the anteroposterior axis A, as shown in FIGS. 3, 8, or 9.

Similarly, the mandibular tray (3) is configured to cover at least one of the user's premolars (PI), molars (MI), and/or canines (CI), right (D) or left (G), of the lower arch (5), also referred to as the mandible (5).

According to the present invention, the upper tray (2) and the mandibular tray (3) are manufactured by thermoforming a mold of the dental impression or by stereolithography so as to perfectly conform to and cover the patient's teeth. As a result, positioning and placing the trays (2 and 3) in the mouth is straightforward with no risk of error, providing a solid mechanical anchoring around or against each tooth.

As illustrated in FIGS. 3 to 5, the upper tray (2) works in conjunction with the mandibular tray (3) via at least one connecting element (6). The connecting element (6) is deployed either on the vestibular faces or on the palatal/lingual faces of the patient's teeth.

In the present invention, the “vestibular face” corresponds to the outward-facing surface of each tooth against the cheek.

Similarly, the “palatal face” corresponds to the inward-facing surface of the user's maxillary, or upper, teeth oriented toward the palate.

Likewise, the “lingual face” corresponds to the inward-facing surface of the user's mandibular, or lower, teeth that are normally in contact with the tongue.

In other words, according to the invention, the connecting element (6) may be in contact either with the mucosa of the patient's cheeks or, conversely, with the patient's tongue and palate.

According to the invention, the connecting element (6) comprises the combination of a peg (7), fixed to either the upper tray (2) or the mandibular tray (3), and a fin (8) with an opening (81) that is fixed to the other tray, respectively (3) or (2).

According to the invention, the connecting element (6) preferably consists of at least one peg (7) coupled with and fitted into the opening (81) of a fin (8).

In a preferred embodiment of the invention shown in FIGS. 3 to 5, the device (1) comprises two connecting elements (6) positioned on both sides of the dental arch, in a plane parallel to longitudinal axis (L), fitting between the upper tray (2) and the mandibular tray (3), preferably on the vestibular faces of the patient's teeth.

These two connecting elements (6), located on the right (D) and left (G) sides of the patient's mouth, stabilize the position of the upper tray (2) relative to the mandibular tray (3) in order to minimize translational displacement of the trays during a shift or horizontal movement of the maxilla (4) relative to the mandible (5), along a horizontal axis (H) shown in FIG. 4.

Advantageously, since both the connecting element (6) and the device (1) are made of a slightly flexible, thin biocompatible material, for example a polyethylene terephthalate resin (PETG) with a thickness of about 0.75 mm, lateral movement of the mandible (5) along the horizontal axis (H) and within the occlusal dental plane remains possible.

According to the invention, the dental occlusal plane comprises or lies parallel to the horizontal axis (H) and passes through the cusp tips of the maxillary teeth. Frontally, the dental occlusal plane must be parallel to the bi-pupillary line, and in profile, it is parallel to a line passing through the infra-orbital rim (under the eye) and the upper border of the patient's auditory canal (ear).

Thus, the flexibility of the material used for the device (1), in particular that of the connecting element (6), ensures patient comfort while maintaining the mechanical correction required to treat Class II or Class III dental malocclusion and, if necessary, dental alignment, while also acting mechanically on OSAS.

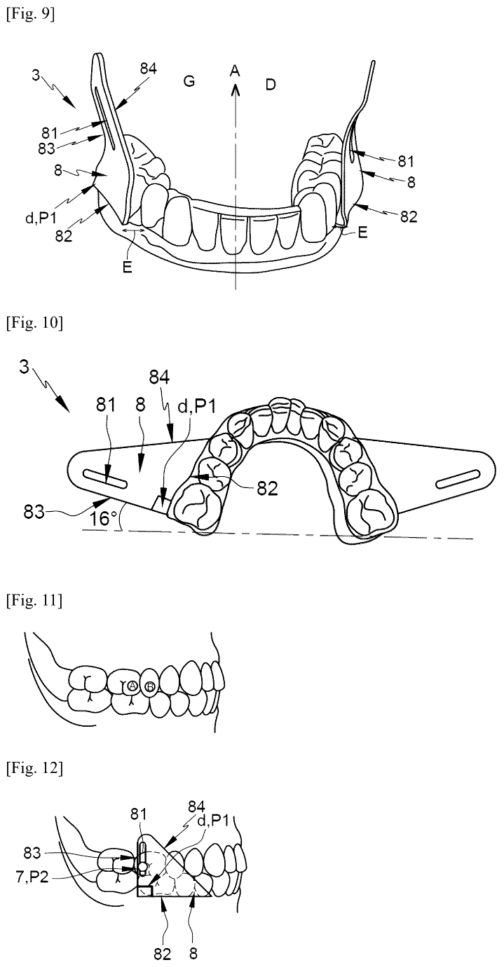

According to another advantage of the invention shown in FIGS. 4 and 9, the connecting element (6) extends toward either the vestibular or palatal/lingual faces of the patient's teeth, with a thickness (E) comprised between 0.8 mm and 1.2 mm, preferably 1 mm.

As shown in FIGS. 4 and 9, when the connecting element (6) is deployed toward the vestibular faces of the user's teeth, that is, in contact with the cheek mucosa, the thickness (E) corresponds to the distance between the tip of the peg (7) that contacts the cheek mucosa and the vestibular-side surfaces of the upper tray (2) and the mandibular tray (3).

In the present invention, the thickness (E) is defined as the distance between the tip of the peg (7), which may contact the cheek mucosa or be oriented toward the patient's tongue or palate, and the surface of the upper tray (2) or the mandibular tray (3) facing, respectively, the vestibular or palatal/lingual sides of the teeth.

Consequently, compared with rod-based devices, the device (1) occupies a minimal volume in the mouth, thereby reducing the risk of cuts or injuries to the cheeks, lips, tongue, or palate. The device (1) significantly improves intraoral comfort for patients while ensuring effective treatment of Class II or Class III dental malocclusion and permitting its combination with mechanical dental alignment therapy via the trays, thereby also treating OSAS.

More specifically, in one particular embodiment of the device (1), the treatment action for Class II or Class III dental malocclusion, together with intraoral comfort, is provided by the connecting element (6), which comprises or consists of a peg (7), fixed to one tray (2 or 3) and coupled with a fin (8) having an opening (81) that is fixed to the other tray (respectively 3 or 2), as shown in FIGS. 3 to 5.

According to the present invention, and as shown in FIG. 7, said peg (7) projects or extends in a direction perpendicular to the vestibular faces or, alternatively, perpendicular to the palatal or lingual faces of the patient's teeth.

In a preferred embodiment, illustrated in the figures, the peg (7) extends in a direction perpendicular to the vestibular faces of the teeth; in other words, in this configuration, the connecting element (6) contacts the patient's cheek mucosa on the outer side of the dental arch.

In another embodiment, not illustrated, said peg (7) extends in a direction perpendicular to the palatal or lingual faces of the teeth; in other words, in this configuration, the connecting element (6) contacts the patient's palate and tongue on the inner side of the dental arch.

More precisely, said peg (7) of device (1) according to the invention is configured to pass through the opening (81) of the fin (8) and be laterally locked therein.

In other words, once inserted and engaged in the opening (81) of the fin (8), the peg (7) is configured to move longitudinally within the opening (81) of the fin (8) while remaining laterally locked.

Longitudinal movement of the peg (7) within the opening (81) enables treatment of Class II or Class III dental malocclusion and mechanical action on OSAS, while lateral locking ensures that the upper tray (2) and the mandibular tray (3) remain connected within the oral cavity, even when the mouth is opened and the jaws are parted.

In a preferred embodiment of the device (1), the peg (7) has a generally mushroom-shaped configuration. In this configuration, the peg (7) comprises a base (71) and a head (72). The base (71) is joined to, or integrated with, either the upper tray (2) or the mandibular tray (3), while the head (72) is designed to engage within the opening (81) of the fin (8) thereby locking the peg (7) transversely within the opening (81).

For example, in one embodiment illustrated in FIG. 6, for a non-monolithic tray design, the base (71) of the peg (7) is secured to one of the trays (2 or 3) by means of a preformed receiving recess (73). The attachment of the base (71) of the peg (7) to one of the trays (2 or 3) may be achieved by any technique known to a person skilled in the art, such as adhesive bonding.

However, other shapes of the peg (7) may be used by a person skilled in the art, provided they allow insertion into the opening (81) and at least partial transverse locking.

In another preferred embodiment illustrated in FIGS. 3 and 7, the peg (7) forms a monolithic piece with either the upper tray (2) or the mandibular tray (3), fabricated for example by stereolithography. This increases the rigidity, strength, and mechanical stability of the peg (7) relative to the upper tray (2) or the mandibular tray (3), and prevents any risk of detachment, even during jaw movement, while also reducing the number of manufacturing steps of the device (1).

In another embodiment illustrated in FIGS. 4 to 6, said peg (7) is fixed to either the upper tray (2) or the mandibular tray (3) after fabrication of the tray. This allows precise adjustment or modification of the position of the peg (7) on the upper tray (2 or 3) without requiring a new tray to be made for the user.

Any biocompatible means known to those skilled in the art for fixing the peg (7) to either the upper tray (2) or the mandibular tray (3) may be employed, provided it does not significantly increase the overall thickness of the device (1).

According to the invention, the peg (7) is configured to pass through the opening (81) of the fin (8) and to move longitudinally therein in order to treat dental malocclusion and/or OSAS.

As shown in FIGS. 8 and 9, the connecting element (6), in addition to the peg (7), further comprises or consists of a substantially triangular fin (8) having an opening (81) and extending vertically between the upper tray (2) and the mandibular tray (3), in a plane parallel to the vestibular or palatal/lingual faces of the patient's teeth.

Thus, in the device (1), the upper tray (2) is connected to the mandibular tray (3) by the connecting element (6). More specifically, the upper tray (2) is secured to the mandibular tray (3) through insertion of the peg (7) into the opening (81) of the fin (8). The opening (81), which is substantially oblong, extends longitudinally between the trays (2 and 3). Consequently, insertion of the peg (7) into the opening (81) allows it to move longitudinally in a direction parallel to the outer or inner surface of the patient's dental arch, that is, along the vestibular or palatal and lingual faces of the teeth.

Advantageously, the oblong opening (81) extends longitudinally between the trays (2 and 3) in a direction perpendicular to the occlusal plane and parallel to the horizontal axis (H), thereby allowing maintenance of mandibular propulsion or retropropulsion during both opening and closing movements of the mouth.

Thus, in the device (1) of the invention, the connecting element (6), that is, the engagement of the peg (7) within the opening (81) of the fin (8), mechanically treats Class II or Class III dental malocclusion. More specifically, movement of the peg (7) within the opening (81) enables mandibular propulsion (advancement) or, conversely, corrective retropropulsion (retraction), even during or after mouth opening.

In a preferred embodiment of the device (1) shown in FIGS. 9, 10, and 12, the substantially triangular fin (8) comprises a leg (82), the entire length of which serves as an anchoring base (82) on either the upper tray (2) or the mandibular tray (3). Securing the full length of the leg (82) of the fin (8) to one of the trays (2 or 3) provides sufficient mechanical cohesion to prevent the fin (8) from detaching from the trays, even when the patient opens the mouth while wearing the device (1).

In one embodiment of the device (1), the fin (8) is manufactured separately from the tray (2 or 3) to which it is attached. In that case, the fin (8) is secured to the corresponding tray (2 or 3) by its anchoring base (82) after individual fabrication of the tray.

In a preferred embodiment of the device (1), shown in FIGS. 9 and 10, the fin (8) having an opening (81) forms a monolithic piece with either the upper tray (2) or the mandibular tray (3). A tray provided with an integral fin (8) of monolithic construction offers greater mechanical resistance to mouth-opening and closing movements and prevents detachment of the fin (8) from the tray to which it is fixed.

Preferably, as illustrated in FIGS. 8, 9, 10, and 12, the fin (8) is substantially in the shape of a right triangle with two legs (82, 83) adjacent to a substantially right angle (d), one of which serves as the anchoring base (82) and is connected to the other by a hypotenuse (84). In this configuration, the hypotenuse (84) of the fin (8) extends between the upper tray (2) and the mandibular tray (3); the right angle (d) defines a first anchoring point (P1) on one tray, while the peg (7), inserted into the opening (81), defines a second fixing point (P2) with the fin (8) on the other tray (3 or 2, respectively).

More specifically, as illustrated in FIG. 12, the opening (81) of the fin (8) extends along a plane and direction substantially parallel to that of side (83), thereby allowing longitudinal translational movement of the peg (7) within the opening (81). This longitudinal movement acts mechanically to correct the offset between the upper and lower arches, thereby treating dental malocclusion.

In a preferred embodiment illustrated in FIG. 11, the anchoring point (P1) or fixing point (P2) of the peg (7) is located on one of trays (2 or 3) at the disto-or mesio-vestibular cusp of the first molar (point A), or at the vestibular face of the second premolar (point B).

Depending on the class of malocclusion to be treated, the practitioner selects the position of the patient's anchoring point (P1) or fixing point (P2) required to mechanically correct mandibular advancement relative to the maxilla.

In a preferred embodiment of device (1) of the invention, the side (83) of the fin (8), adjacent to the right angle (d) and not serving as the anchoring base (82), defines an angular offset comprised between 0° and 20°, preferably about 16°, relative to the right-angle anchoring point (P1) on the mandibular tray (3) or the upper tray (2), respectively.

The choice of the angular offset, together with the position of the anchoring point (P1) or fixing point (P2) and the extent of the opening (81) of the fin (8), allows precise adjustment of mandibular advancement or retrusion to effectively treat the patient's Class II or Class III dental malocclusion.

For example, as illustrated in FIG. 12, with a 16 ° angular offset and anchoring of the peg (7) to the maxilla at the fixing point (P2) on the disto-vestibular cusp of the first molar, and with an oblong opening (81) substantially parallel to side (83), a Class I molar normocclusion can be achieved, corresponding to a correction by mandibular advancement relative to the maxilla.

As shown in FIG. 11, this mandibular correction can be further accentuated by shifting the fixing point (P2) toward the mesio-vestibular cusp of the first molar (point A) or toward the vestibular face of the second premolar (point B).

Thus, the device (1) offers numerous possibilities, enabling a skilled person to correct abnormal contact between the patient's maxillary and mandibular teeth in Class II or Class III dental malocclusion, while also allowing treatment of dental misalignment and mechanical action on OSAS.

In another embodiment of the device (1), the upper tray (2), the mandibular tray (3), and the fin (8) each have a thickness less than or equal to 0.75 mm. In other words, the thin contact surfaces of the trays, which rest against the teeth, ensure intraoral comfort for the patient.

To minimize the intraoral bulk of the device (1) in the patient's mouth, the skilled person selects materials for the upper tray (2), the mandibular tray (3), the fin (8), and the peg (7) that are sufficiently strong, as thin as possible, biocompatible, safe for the patient, and preferably transparent, allowing simultaneous treatment of dental malocclusion and alignment defects.

According to the invention, the device (1) may be produced by machining, filament deposition, milling, or any other means known to those skilled in the art.

Preferably, device (1) is made by thermoforming or stereolithography for a simple, minimal-step, custom fabrication prior to placement in the patient's mouth.

With its connecting element (6) integrated in direct continuity with the trays (2 and 3), the device (1) limits labial-zone interference, allows mouth opening without any appliance between the upper and lower lips, and prevents external sub-labial deformation.

Furthermore, the device (1) is minimally bulky in the mouth, as the connecting element (6) has a thickness similar to, or equal to, that of the trays (2 and 3), ensuring patient comfort.

In addition, the device (1) enables corrective mandibular advancement (propulsion) or retrusion (retropropulsion) and treatment of Class II or Class III dental malocclusion even during mouth opening, thanks to the openings (81) formed in the fins (8), while also permitting lateral jaw flexibility.

More specifically, in the device (1), mandibular propulsion or retropropulsion is achieved by the connecting element (6), which is formed as an extension integral with the upper tray (2) and the mandibular tray (3). The configuration of the connecting element (6), with its anchoring base (82) extending from the tray and the peg (7) translating within the opening (81) of the (8), provides corrective action while maintaining propulsion and retropropulsion even during mouth opening.

Thus, the device (1), through its connecting element (6) and specific configuration, positions the mandible for correction of Class II or Class III dental malocclusion, while remaining compatible with simultaneous dental alignment correction via the trays (2 and 3) and mechanical treatment of OSAS.

Accordingly, the device (1), by virtue of its structural design, treats Class II or Class III dental malocclusion through the specific connecting element (6), while also enabling dental alignment correction via the trays (2 and 3), both in closed and open mouth positions, without injuring the mucosa, tongue, or palate, or obstructing the labial zone. The disadvantages of rod-or spring-based devices are thereby overcome, as the device is compact, simple in design, and low in production cost.

1. An orthodontic device for the treatment of dental alocclusion, correction of dental alignment, and treatment of obstructive sleep apnea syndrome, the orthodontic device comprising:
an upper dental tray and a mandibular dental tray, each configured to conform at least partially to a dental arch of a user;
the upper dental tray being configured to cover at least one premolar and/or molar on each side of an upper arch of the user;
the mandibular dental tray configured to cover at least one premolar and/or molar and/or canine on each side of a lower arch, or mandible of the user;
the upper dental tray cooperating with the mandibular dental tray via at least one connecting element positioned along vestibular faces of teeth of the user or along palatal and lingual faces of the teeth of the user;
the connecting element comprising the association of a peg, integral with either the upper dental tray or the mandibular dental tray, and a fin having an opening, integral respectively with the mandibular dental tray (3) or the upper dental tray (2);
the peg, projecting in a plane perpendicular to the vestibular faces or to the palatal and lingual faces of the teeth of the user, being configured to pass through the opening of the fin and to be at least partially laterally retained therein;
the fin being substantially triangular;
the fin being oriented vertically between the upper dental tray and the mandibular dental tray in a plane parallel to the vestibular faces or to the palatal and lingual faces of the teeth of the user;
the opening extending between the upper dental tray and the mandibular dental tray along a longitudinal axis; and
the upper dental tray cooperating with the mandibular dental tray by insertion of the peg into the opening of the fin, thereby allowing longitudinal translational movement of the peg within the opening along a direction substantially parallel to the longitudinal axis, and at least partial blocking of the movement of the peg within the opening along a horizontal axis during lateral movement of a maxilla relative to a mandible of the user.

2. The orthodontic device according to claim 1, comprising two connecting elements positioned on either side of the dental arch, in a plane parallel to longitudinal axis.

3. The orthodontic device according to claim 1, wherein the at least one connecting element extends toward either the vestibular faces or the palatal and lingual faces of the teeth of the user with a thickness in a range of from 0.8 mm to 1.2 mm.

4. The orthodontic device according to claim 1, wherein
the fin is substantially triangular;
the fin comprises a side whose entire length serves as an anchoring base on either the upper dental tray or the mandibular dental tray; and
the fin comprises another side whose end is secured, by insertion of the peg into the opening, to the mandibular dental tray or, respectively, to the upper dental tray.

5. The orthodontic device according to claim 1, wherein
the fin is substantially in the form of a right triangle comprising a hypotenuse connected by two legs meeting at a substantially right angle, one of the legs serving as an anchoring base;
the hypotenuse extends between the upper dental tray and the mandibular dental tray; and
the right angle defines a first anchoring point on one of the upper dental tray and the mandibular dental tray, while the peg, inserted into the opening, defines a fixing point with the fin on the other of the upper dental tray and the mandibular dental tray.

6. The orthodontic device according to claim 5, wherein the anchoring point or the fixing point is positioned on one of the upper dental tray and the mandibular dental tray at a disto-vestibular cusp or mesio-vestibular cusp of a first molar, or on a vestibular face of a second premolar.

7. The orthodontic device according to claim 5, wherein the leg of the fin that does not serve as anchoring base has an angular offset in a range of from 0°to 20°relative to the right angle anchoring point on the upper dental tray or the mandibular dental tray.

8. The orthodontic device according to claim 1, wherein
the peg forms a monolithic piece with either the upper dental tray or the mandibular dental tray, and/or
the fin with the opening forms a monolithic piece with either the upper dental tray or the mandibular dental tray.

9. The orthodontic device according to claim 1, wherein the upper dental tray, the mandibular dental tray, and the fin each have a thickness less than or equal to 0.75 mm.

10. The orthodontic device according to claim 1, which has been manufactured by thermoforming or by stereolithography.

11. The orthodontic device according to claim 1, wherein the opening is substantially oblong.

12. The orthodontic device according to claim 2, wherein the two connecting elements are positioned along the vestibular faces of the teeth of the user.

13. The orthodontic device according to claim 7, wherein the angular offset of the leg of the fin that does not serve as anchoring base is 16°, relative to the right angle anchoring point on the upper dental tray or the mandibular dental tray.

14. The orthodontic device according to claim 6, wherein the leg of the fin that does not serve as anchoring base has an angular offset in a range of from 0° to 20° relative to the right angle anchoring point on the upper dental tray or the mandibular dental tray.

15. The orthodontic device according to claim 2, wherein the at least one connecting element extends toward either the vestibular faces or the palatal and lingual faces of the teeth of the user with a thickness in a range of from 0.8 mm to 1.2 mm.

16. The orthodontic device according to claim 15, wherein
the fin is substantially triangular;
the fin comprises a side whose entire length serves as an anchoring base on either the upper dental tray or the mandibular dental tray; and
the fin comprises another side whose end is secured, by insertion of the peg into the opening, to the mandibular dental tray or, respectively, to the upper dental tray.

17. The orthodontic device according to claim 2, wherein
the fin is substantially triangular;
the fin comprises a side whose entire length serves as an anchoring base on either the upper dental tray or the mandibular dental tray; and
the fin comprises another side whose end is secured, by insertion of the peg into the opening, to the mandibular dental tray or, respectively, to the upper dental tray.

18. The orthodontic device according to claim 3, wherein
the fin is substantially triangular;
the fin comprises a side whose entire length serves as an anchoring base on either the upper dental tray or the mandibular dental tray; and
the fin comprises another side whose end is secured, by insertion of the peg into the opening, to the mandibular dental tray or, respectively, to the upper dental tray.

19. The orthodontic device according to claim 2, wherein
the fin is substantially in the form of a right triangle comprising a hypotenuse connected by two legs meeting at a substantially right angle, one of the legs serving as an anchoring base;
the hypotenuse extends between the upper dental tray and the mandibular dental tray ; and
the right angle defines a first anchoring point on one of the upper dental tray and mandibular dental tray, while the peg, inserted into the opening, defines a fixing point with the fin on the other of the upper dental tray and the mandibular dental tray.

20. The orthodontic device according to claim 3, wherein
the fin is substantially in the form of a right triangle comprising a hypotenuse connected by two legs meeting at a substantially right angle, one of the legs serving as an anchoring base;
the hypotenuse extends between the upper dental tray and the mandibular dental tray; and
the right angle defines a first anchoring point on one of the upper dental tray and the mandibular dental tray, while the peg, inserted into the opening, defines a fixing point with the fin on the other of the upper dental tray and the mandibular dental tray.
